(19) United States
(12) Patent Application Publication
(10) Pub. No.: US 2023/0301985 A1
(43) Pub. Date: Sep. 28, 2023

(54) MOVEMENT DISORDERS

(71) Applicant: Mind Medicine, Inc., New York, NY (US)

(72) Inventors: Robert BARROW, Madison, WI (US); Daniel R. KARLIN, New York, NY (US); Gennady SMAGIN, Newark, DE (US)

(73) Assignee: Mind Medicine, Inc., New York, NY (US)

(21) Appl. No.: 18/197,892

(22) Filed: May 16, 2023

Related U.S. Application Data
(63) Continuation-in-part of application No. 17/734,102, filed on May 1, 2022.
(60) Provisional application No. 63/183,771, filed on May 4, 2021.

(51) Int. Cl. A61K 31/48; A61K 31/36; A61P 25/00; A61K 31/437

(52) U.S. Cl. CPC A61K 31/48; A61K 31/36; A61P 25/00; A61K 31/437

(57) ABSTRACT

A method of treating movement disorders, by administering an effective amount of a psychedelic to an individual having a movement disorder, and treating the movement disorder.

### BACKGROUND OF THE INVENTION

### 1. Technical Field

The present invention relates to compositions and methods for treatments of movement disorders. More specifically, the present invention relates to methods of treating movement disorders with psychedelics.

### 2. Background Art

Tremors are involuntary, rhythmic, and oscillatory movement of a body part and are the most common movement disorder. They are caused by alternating or synchronous contractions of antagonistic muscles. Tremors can occur when the body is in a rest state (the body part is fully supported, relaxed, or not voluntarily activated) or in an action state (during voluntary movement, during a particular posture, or during muscle contraction against a stationary object).

Parkinson's disease is a common rest tremor and also causes stiffness, slow decreased movement, and imbalance. Rest tremor is usually treated with antiparkinson agents such as anticholinergic drugs, amantadine, dopamine agonists, levodopa, or zonisamide.

Essential tremor (ET) is a common action tremor, and most often affects hands and arms and can also affect the head, voice, face, or trunk. ET is activated by voluntary movement or when the arms are held in a fixed posture against gravity. ET has a strong genetic component and runs in families. 1 percent of the worldwide population suffers from ET and 5 percent of adults over 60 years old. Tremors can be exacerbated due to anxiety, excitement, or other adrenergic stimulation. Action tremors such as ET can be treated with propranolol or primidone as the two primary treatments, or with topiramate, gapapentin, botulinum toxin injections, as well as deep brain stimulation and unilateral thalamotomy. Primidone has acute adverse reactions (sedation, drowsiness, fatigue, depression, nausea, vomiting, ataxia, malaise, dizziness, unsteadiness, confusion, vertigo, and an acute toxic reaction), and propranolol has chronic side effects (lightheadedness, fatigue, impotence, and bradycardia). Other treatments of movement disorders include deep brain stimulation, physical therapy, and occupational therapy.

Tremors are also a side effect of certain drugs. Drugs that can cause tremors include cancer medicines such as thalidomide and cytarabine, seizure medicines such as valproic acid and sodium valproate, asthma medicines such as theophylline and albuterol, immune suppressing medicines such as cyclosporine and tacrolimus, mood stabilizers such as lithium carbonate, stimulants such as caffeine and amphetamines, antidepressant drugs such as selective serotonin reuptake inhibitors (SSRIs) and tricyclics, heart medicines such as amiodarone, procainamide, and others, certain antibiotics, certain antivirals, such as acyclovir and vidarabine, alcohol, nicotine, certain high blood pressure drugs, epinephrine and norepinephrine, weight loss medicine (tiratricol), too much thyroid medicine (levothyroxine), or tetrabenazine, a medicine to treat excessive movement disorder. Individuals are embarrassed by this side effect but it will not go away unless they stop taking the drug or dosing can be adjusted.

Psychedelics are substances capable of inducing exceptional subjective effects such as a dream-like alteration of consciousness, affective changes, enhanced introspective abilities, visual imagery, pseudo-hallucinations, synaesthesia, altered temporal and special perception, mystical-type experiences, disembodiment and ego dissolution (Liechti, 2017; Passie, Halpern, Stichtenoth, Emrich & Hintzen, 2008). Psychedelics can be used to assist psychotherapy for many indications including anxiety, depression, addiction, personality disorder and others and can also be used to treat other disorders such as cluster headache and migraine and others (Passie et al., 2008; Hintzen et al., 2010; Nichols, 2016; Liechti, 2017).

There remains a need for an effective treatment for movement disorders.

### SUMMARY OF THE INVENTION

The present invention provides for a method of treating movement disorders, by administering an effective amount of a psychedelic to an individual having a movement disorder and treating the movement disorder.

### DESCRIPTION OF THE DRAWINGS

Other advantages of the present invention are readily appreciated as the same becomes better understood by reference to the following detailed description when considered in connection with the accompanying drawings wherein:

FIG. 1 is a graph of locomotor activity/movement data from a harmaline-induced tremor model in rats with propranolol as a positive control;

FIG. 2 is a graph of tremor power ratio data from a harmaline-induced tremor model in rats with propranolol as a positive control;

FIG. 3 is a graph of tremor power ratio data from a harmaline-induced tremor model in rats treated with LSD and R-MDMA; and

FIG. 4 is a graph of brain harmaline levels in rats treated with LSD and R-MDMA.

### DETAILED DESCRIPTION OF THE INVENTION

The present invention provides for a method of treating movement disorders, by administering an effective amount of a psychedelic to an individual having a movement disorder and treating the movement disorder.

“Movement disorder” as used herein, can refer to any neurological condition that results in abnormal increased movements in the body, reduced movements, or slow movements. The movements can affect one part of the body or multiple parts. The movements can be voluntary or involuntary. The movements can be triggered by specific situations. The movement disorders treated herein can include, but are not limited to, ataxia, cervical dystonia, chorea, dystonia, functional movement disorder, Huntington's disease, multiple system atrophy, myoclonus, Parkinson's disease, Parkinsonism, progressive supranuclear palsy, restless legs syndrome, spasticity, tardive dyskinesia, tic disorders (including Tourette syndrome), tremor (including essential tremor), or Wilson's disease. The movement disorder can be a rest tremor or action tremor. The movement disorder can also be caused by side effects of drugs the individual is taking or by a stressful life event. The movement disorders can be caused by brain injury (i.e., head trauma, infection, inflammation, metabolic disturbances, toxins) or unintended side effects of medications, or they can be a symptom of underlying diseases or conditions, including genetic disorders.

The psychedelics in the present invention can be, but are not limited to, lysergic acid diethylamide (LSD), psilocybin, psilocin, mescaline, 3,4-methylenedioxymethamphetamine (MDMA, including its individual enantiomer forms R-MDMA or S-MDMA), 5-methoxy-N,N-dimethyltryptamine (5-MeO-DMT), dimethyltryptamine (DMT), 2,5-dimethoxy-4-iodoamphetamine (DOI), 2,5-dimethoxy-4-bromoamphetamie (DOB), salts thereof, tartrates thereof, solvates thereof, isomers thereof, analogs thereof, homologues thereof, or deuterated forms thereof. Preferably, the dose of the psychedelic is one that provides a meaningful clinical effect or can be a dose that is a perceptual dose or subperceptual dose. The psychedelic drug can be administered as a single dose or as repeat doses over multiple days, weeks, months, or years. A dose of 0.05-1 mg (10-1000 μg) can be used of LSD. Psilocybin can be dosed at 1-50 mg, psilocin can be dosed at 1-100 mg, mescaline can be dosed at 10-1000 mg, 5-MeO-DMT can be dosed at 0.2-20 mg, DMT can be dosed at 10-100 mg, DOI can be dosed at 0.1-10 mg, and DOB can be dosed at 0.1-5 mg. Effects of a single dose of the psychedelic drug can last 1-12 hours after administration, and the individual can be supervised by medical personnel such as a psychiatrist during this time. If lower doses are given, medical supervision can be unnecessary.

Mechanistically, psychedelics act as nonspecific serotonin agonists. LSD potently stimulates the 5-HT_{2A} receptor but also 5-HT_{2B}/C, 5-HT₁ and D₁₋₃ receptors (Rickli et al., 2016). Serotonergic psychedelics have their psychoactive/hallucinogenic effects by agonism at the serotonin 5-HT_{2A} receptor. LSD induces its psychedelic effects in humans primarily via stimulation of the 5-HT_{2A} receptor (Kraehenmann et al., 2017; Preller et al., 2017; Barrett et al., 2018). Psilocybin (3-(2-dimethylaminoethyl)-1H-indol-4-yl] dihydrogen phosphate) is a psychedelic drug that is produced by psilocybin mushrooms, such as, but not limited to, P. azurescens, P. semilanceata, and P. cyanescens. Psilocin, the active metabolite of psilocybin, inhibits the 5-HT transporter (SERT) whereas LSD stimulates D₁₋₃ receptors but does not interact with the SERT (Rickli et al., 2016). In contrast to LSD, psilocybin and mescaline show no affinity for D₂ receptors. The potent dopaminergic receptor agonist properties of LSD have been linked to delayed LSD effects that are possibly distinct from other hallucinogens and possibly more stimulant-like (Mittman et al., 1991; Marona-Lewicka et al., 2005; Marona-Lewicka et al., 2007; Nichols, 2016). LSD and the tryptamines DMT and psilocin are potent agoinsts at serotonin 5-HT1 receptors while other hallucinogens such as mescaline exhibit low potency at this receptor (Rickli et al., 2016). While no clinical studies have clearly documented a role for the 5-HT1 receptor (Strassman, 1996; Nichols, 2016) in the action of psychedelics, differences between substances may exist. SERT inhibition (Rickli et al., 2016) and increases in serotonin by psilocybin may be associated with greater serotonergic toxicity including nausea and vomiting when psilocybin is used compared to other psychedelics with no interaction with the SERT. Mescaline binds in a similar concentration range to 5-HT_{2A}, 5-HT_{1A} and adrenergic α_{2A} receptors (Rickli et al., 2016).

In treating movement disorders, the effect of the psychedelics can be a CNS effect, direct peripheral effect, or combination of both. Movement disorders can be caused by central or peripheral problems in the body. As with the treatment of other disorders, there are multiple schools of thought as to how psychedelics exert their effects. There is evidence that they are effective via psychological mechanisms, based in the experience of the direct effects of the drug on 5HT_{2A} receptors. There is also evidence that they have direct neurobiological effects in the brain, and that they enhance whole brain connectivity and introduce a new degree of neuroplasticity in cortical neurons. Further, there is evidence of effect driven by central nervous system binding outside of the brain in addition to peripheral nervous system effects outside of the spinal cord. There has been some review of use of psychedelics in treating FND (functional neurological disorder), however, mixed results were seen in various studies and some thought psychedelics would exacerbate symptoms (Butler, et al. Ther Adv Psychopharmacol. 2020 May 11). Though the exact mechanism of action against movement disorders remains not fully specified, it is likely some combination of these putative mechanisms, and the present invention provides unexpected results in treating movement disorders.

In the method of the present invention, treating the movement disorder can include reducing and/or eliminating undesired movement or tremors, or returning any abnormal movement to a normal state. The method can further include reducing anxiety and therefore reducing abnormal movement due to anxiety. The method can further reduce tremors due to side effects of the drugs that the individual is taking.

The compounds of the present invention are administered and dosed in accordance with good medical practice, considering the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

In the method of the present invention, the compounds of the present invention can be administered in various ways. It should be noted that they can be administered as the compound and can be administered alone or as an active ingredient in combination with pharmaceutically acceptable carriers, diluents, adjuvants, and vehicles. The compounds can be administered orally, transcutaneously, subcutaneously or parenterally including sublingual, buccal, inhalation, intravenous, intramuscular, and intranasal administration. The patient being treated is a warm-blooded animal and, in particular, mammals including man. The pharmaceutically acceptable carriers, diluents, adjuvants, and vehicles as well as implant carriers generally refer to inert, non-toxic solid or liquid fillers, diluents or encapsulating material not reacting with the active ingredients of the invention.

The doses can be single doses or multiple doses or a continuous dose over a period of several hours, days, weeks, months, or years.

When administering the compound of the present invention orally, it will generally be formulated in an immediate release capsule, immediate release tablet, modified release capsule or tablet (including enteric coatings), solution, or suspension. When administering the compound of the present invention parenterally, it will generally be formulated in a sublingual or buccal orally dissolving tablet, dissolving film, intranasal powder, intranasal solution, inhaled powder, inhaled solution, transdermal patch, transdermal patch with microneedles or other permeation enhancers, or as a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

The invention is further described in detail by reference to the following experimental examples. These examples are provided for the purpose of illustration only and are not intended to be limiting unless otherwise specified. Thus, the invention should in no way be construed as being limited to the following examples, but rather, should be construed to encompass any and all variations which become evident as a result of the teaching provided herein.

### Example 1

### Experimental Procedures

This experiment was performed in rats (CD/SD), source Charles River Labs, age or weight: 180-200 grams on arrival, sex: male. Randomization: Animals were assigned randomly to treatment groups.

Route of administration for harmaline was IP, for TA was PO. Dose volume was 3 mL/kg. Formulation(s): harmaline: 0.9% saline; TA: PBS is used as a vehicle for TA. Dose Levels: harmaline: 30 mg/kg; LSD-100 ug/kg PO, R-MDMA-2 mg/kg. Dose Frequency: 1×. Study duration: 1 day. Pretreatment time (up to 2 hours): 15 minutes. Number of Groups: 4. Number of animals per group: 10. Total number of animals: 40. The study design is summarized in TABLE 1.

TABLE 1

| | | | | DOSE | | |
|---|---|---|---|---|---|---|
| | | | | TA/ | ROUTE | |
| GROUP | | GROUP | DAYS OF | HARMALINE | TA/ | EVALUATIONS/ |
| # | TREATMENT | SIZE | DOSING | (MG/KG) | HARMALINE | ENDPOINTS |
| 1 | Vehicle/ | 10 | 1x | 0/0 | PO/IP | Tremor power |
| | Saline | | | | | ratio |
| 2 | Vehicle/ | | | 0/30 | | (0-60 minutes |
| | Harmaline | | | | | post harmaline) |
| 3 | TA1(LSD)/ | | | 0.1/30 | | Locomotor |
| | Harmaline | | | | | activity |
| 4 | TA2(R-MDMA | | | 2/30 | | Harmaline brain |
| | HCL)/ | | | | | concentration |
| | Harmaline | | | | | (Groups 2-4) |

Methods

Male CD/SD rats (CRL) were used, 180-200 g at time of arrival. They were acclimated for 5-7 days prior to testing. Each rat was tested only once and then euthanized for brain tissue collection.

Drugs

Harmaline (harmaline HCl dihydrate; Sigma H1392-1) was obtained by Melior and formulated using a free-base correction factor of 1.33 in saline at 10 mg/mL (13.3 mg/mL with correction) and administered at 3 ml/kg by IP route.

Saline (0.9% NaCl) was formulated at Melior using sterile filtered water.

Testing Procedure

For groups 1-4 each animal received two treatments 15 minutes apart (see TABLE 1). The first treatment was either TA or vehicle. The second treatment was either harmaline or saline. Immediately after the second dosing, the animal was placed into the actimeter (BASi Force Plate Actimeter). The actimeter consists of an enclosed container (41.6×41.6 cm) containing a rigid plate that rests on four force transducers placed at each corner. The transducers measure the distribution of forces exerted by the animal while moving about.

Four treatment groups were evaluated in this study, with the test agent, dose levels, and N per group listed in TABLE 1. Motor activity was monitored for 60 minutes after administration of the second test agent (harmaline or saline). TPR was calculated as the average of the tremor during or a 10-minute period for six successive 10 minute periods beginning at 0 minutes after harmaline administration (i.e., from 0 minutes to 60 minutes).

Tremor power was evaluated using the ratio TPR=(P2)/(P1+P3), where Pn=integrated power over (1) 1.5-8 Hz, (2) 8-13 Hz, and (3) 13-25 Hz (the frequency range may be optimized depending on the actual data at the time of testing). The Tremor Power Ratios were evaluated by 3-factor ANOVA (Time, Frequency, Treatment)

As part of the analysis, brain levels of harmaline were measured. Outliers with low brain harmaline levels (greater than 2 standard deviations) were excluded from analysis.

Tissue Samples and Euthanasia

Brains were harvested from all animals treated with harmaline at 60 minutes (end of testing). The brains of those rats were removed, divided into two hemispheres along the mid-sagittal plane, weighed, and rapidly frozen on dry ice. The concentrations of harmaline were evaluated by HPLC-mass spectroscopy in the left hemispheres.

Results and Discussion

The harmaline-induced model is a classical animal model of action tremor. (Pan, et al. Animal models of tremor: relevance to human tremor disorders. Tremor Other Hyperkinet Mov. 2018; 8.) A single dose of harmaline can induce action tremor by enhancing the coupling between the inferior olivary (10) neurons. Harmaline-induced tremor is predominantly an action tremor that responds to propranolol, primidone, and alcohol. Therefore, harmaline-induced tremor has long been postulated to be an animal model of ET. Harmaline belongs to a group of naturally occurring compounds, called β-alkaloids. In ET patient blood and brain, increased harmaline-related β-alkaloids, such as harmane, have been observed, suggesting that environmental factors can contribute to oscillatory activities in the olivocerebellar system in ET patients. Harmaline has been shown to induce action tremor in a wide variety of animals, including mice, rats, cats, monkeys, and pigs, suggesting an evolutionarily conserved olivocerebellar circuit for tremor generation. Harmaline-induced tremor model has been tested in pre-clinical studies for ET for the development of a gap junction blocker, carbenoxolone, and a T-type calcium channel blocker. Translational validity of this model is confirmed in clinical studies. Therefore, results obtained in this model can predict clinical efficacy of tested compound in humans.

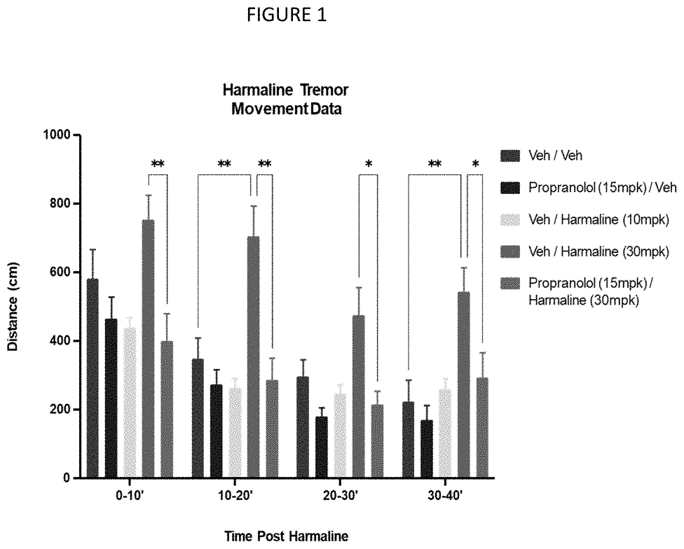

FIG. 1 shows an example of locomotor activity/movement data from the harmaline-induced tremor model in rats with Propranolol (15 mg/kg, IP) used as a positive control. Measurements were done every 10 minutes over 60 minutes post harmaline administration. 10 rats per group.*-P<0.05, **-P<0.01, 3-factor ANOVA (Time, Frequency, Treatment).

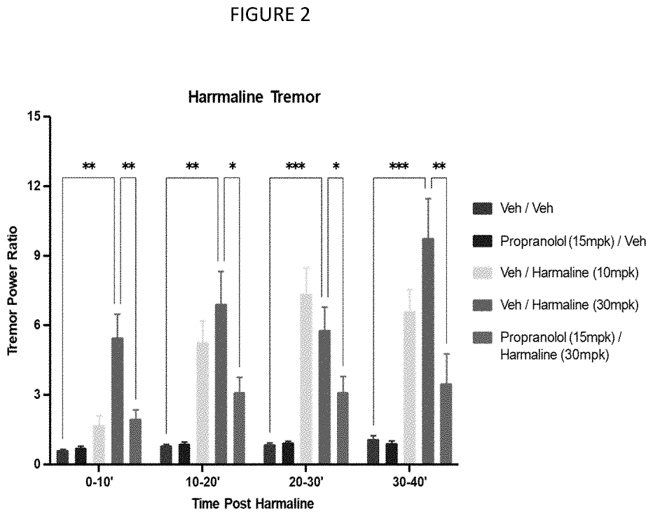

FIG. 2 shows an example of tremor power ratio data from the harmaline-induced tremor model in rats with Propranolol (15 mg/kg, IP) used as a positive control. Measurements were done every 10 minutes over 60 minutes post harmaline administration. 10 rats per group.*-P<0.05, **-P<0.01, ***-P<0.001 3-factor ANOVA (Time, Frequency, Treatment).

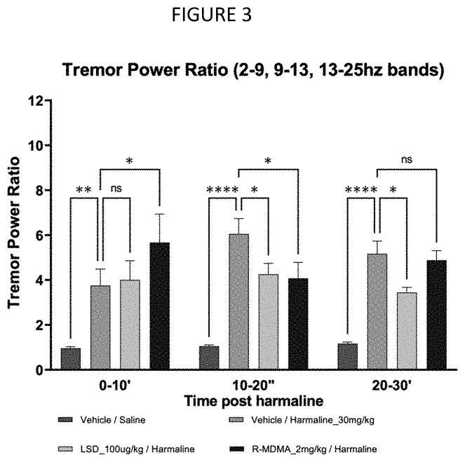

FIG. 3 shows an example of tremor power ratio data from the harmaline-induced tremor model in rats treated with the LSD and R-MDMA. Measurements were done every 10 minutes over 60 minutes post harmaline administration. 10 rats per group.*-P<0.05, **-P<0.01, ****-P<0.0001, ns-not significant. 2-factor ANOVA (Time, Treatment).

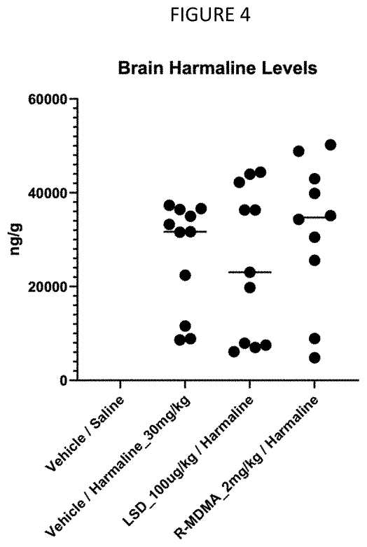

FIG. 4 shows an example of brain harmaline levels in animals from the harmaline-induced tremor model in rats treated with the LSD and R-MDMA presented in FIG. 3. Brains were harvested from all animals treated with harmaline at 60 minutes (end of testing). Harmaline was measured by LC/MS/MS. There is no significant difference between the groups. No outliers were detected or excluded from the analyses.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

### REFERENCES

1. Handforth, A. Harmaline Tremor: Underlying Mechanisms in a Potential Animal Model of Essential Tremor. Tremor and Other Hyperkinetic Movements, pps. 1-14.
2. Kulkarni S K, Kaul P N. Modification by levo-propranolol of tremors induced by harmaline in mice. Experientia 1979: 35:1627-28.
3. Long M A, Deans M R, Paul D L, Connors B W. Rhythmicity without synchrony in the electrically uncoupled inferior olive. J Neurosci. 2002; 22:10898-905.
4. Martin F C, Thu Le A, Handforth A. Harmaline-induced tremor as a potential preclinical screening method for essential tremor medications. Mov Disord. 2005; 20(3):298-305.
5. Paul V. Involvement of beta 2-adrenoceptor blockade and 5-hydroxytryptamine mechanism in inhibition of harmaline-induced tremors in rats. Eur J Pharmacol. 1986; 122:111-5.
6. Sinton C M(1), Krosser B I, Walton K D, Llinás R R. The effectiveness of different isomers of octanol as blockers of harmaline-induced tremor. Pflugers Arch. 1989; 414:3.

What is claimed is:

1. A method of treating movement disorders, including the steps of:
administering an effective amount of a psychedelic to an individual having a movement disorder; and
treating the movement disorder.

2. The method of claim 1, wherein the movement disorder is chosen from the group consisting of ataxia, cervical dystonia, chorea, dystonia, functional movement disorder, Huntington's disease, multiple system atrophy, myoclonus, Parkinson's disease, Parkinsonism, progressive supranuclear palsy, restless legs syndrome, spasticity, tardive dyskinesia, tic disorders, tremor, and Wilson's disease.

3. The method of claim 1, wherein the psychedelic is chosen from the group consisting of lysergic acid diethylamide (LSD), psilocybin, psilocin, mescaline, 3,4-methylenedioxymethamphetamine (MDMA), 5-methoxy-N,N-dimethyltryptamine (5-MeO-DMT), dimethyltryptamine (DMT), 2,5-dimethoxy-4-iodoamphetamine (DOI), 2,5-dimethoxy-4-bromoamphetamie (DOB), salts thereof, tartrates thereof, solvates thereof, isomers thereof, analogs thereof, homologues thereof, or deuterated forms thereof.

4. The method of claim 1, wherein said treating step further includes the step of reducing and/or eliminating undesired movement or tremors.

5. The method of claim 1, wherein said treating step further includes the step of returning abnormal movement to a normal state.

6. The method of claim 1, wherein said treating step further includes the step of reducing anxiety and reducing abnormal movement due to anxiety.

7. The method of claim 1, wherein said treating step further includes the step of reducing tremors due to side effects of drugs that the individual is taking.
